(19) United States
(12) Patent Application Publication
(10) Pub. No.: US 2010/0330555 A1
(43) Pub. Date: Dec. 30, 2010

(54) ACCURACY FLUORESCENCE IN-SITU HYBRIDIZATION ASSAY OF SAMPLES WITH APOPTOTIC CELLS

(75) Inventor: Michael John Gerdes, Albany, NY (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: GENERAL ELECTRIC COMPANY, Schenectady, NY (US)

(21) Appl. No.: 12/491,484

(22) Filed: Jun. 25, 2009

(51) Int. Cl. C12Q 1/68; G01N 33/53; C12M 1/34

(52) U.S. Cl. 435/6; 435/7.2; 435/287.2

(57) ABSTRACT

The present application discloses a process for improving the accuracy of fluorescence in-situ hybridization (FISH) assays in which the sample being assayed is likely to contain cells in apoptosis by excluding these cells from the evaluation of the FISH assay. This is conveniently done by labeling the cells in apoptosis by incorporating labeled nucleotides into the apoptosis typical breaks in their nuclear DNA. The present application also discloses a kit and system adapted for carrying out this process for improving the accuracy of FISH assays.

### BACKGROUND

The subject matter disclosed herein relates generally to fluorescence in-situ hybridization (FISH) assays of samples of cells in which a significant proportion of the cells are in apoptosis. A defining characteristic of cells in apoptosis is that their DNA has begun to degrade. As these cells undergo their programmed cell death they release activated nucleases which attack and fragment their nuclear DNA. A significant proportion of such cells can not be reliably identified from cell morphology. However, FISH assays of such cells are likely to be inaccurate because the of the DNA fragmentation. For example, an apoptotic cell may have contained the DNA sequence targeted by the FISH probes, perhaps a sequence characteristic of a given genetic mutation. But the apoptotic degradation may have cleaved the target sequence leading to a false negative result in the FISH assay.

Cancer cells from either a tumor or a leukemia are frequently the object of FISH assays. For instance the value of certain breast cancer treatments, such as the administration of Herceptin® biologic to a given patient, is assessed by evaluating the HER2 genetic marker. But samples of cancer cells often contain a significant proportion of cells in apoptosis. For instance in myelodysplastic syndromes (MDS) it has been found that the rate of apoptosis is so high as to effectively render the effected cells unable to engage in effective hematopoiesis.

Thus there is a need to more comprehensively adjust FISH assays of cell populations in which a significant proportion of the cells are undergoing apoptosis to take account of sources of error such as a loss of signal from DNA fragmentation than can be done from an observation of cell morphology.

### BRIEF DESCRIPTION

The present invention involves a process for improving the accuracy of fluorescence in-situ hybridization (FISH) assays in which the sample being assayed is likely to contain cells in apoptosis by excluding these cells from the evaluation of the FISH assay. This is conveniently done by labeling the cells that are in some stage of apoptosis and then eliminating them from the FISH analysis. One convenient approach is to outline all the cell nuclei in the sample being evaluated and then not take any account of the presence or absence of a signal from the FISH assay which originates in a nucleus which also displays a signal from the apoptosis assay. The nuclei may be outlined using an intercalating dye. The elimination can by done manually by the observations of a skilled examiner or by automated means such as the processing of digital images.

### DRAWINGS

These and other features, aspects, and advantages of the present invention will become better understood when the following detailed description is read with reference to the accompanying drawings in which like characters represent like parts throughout the drawings, wherein:

FIG. 1A is an image taken by a monochromatic digital camera of the fluorescent signal from Rhodamine conjugated to anti-digoxingenin antibodies associated with a nucleotides incorporated into breaks in the nuclear DNA of a sample of a xenograft of human colon cancer implanted into nude mice.

FIG. 1B is an image taken by a monochromatic digital camera of the fluorescent signal from Cy5 dye conjugated to strepavadin associated with a biotin bearing nucleic acid probe to the centromere of human chromosome 10 hybridized to the same sample as FIG. 1A.

FIG. 1C is an image taken by a monochromatic digital camera of the fluorescent signal from DAPI reacted with the DNA of the same sample as FIG. 1A.

FIG. 2A is an image taken by a monochromatic digital camera of the fluorescent signal from DAPI reacted with the DNA of another sample of the same material as FIG. 1A.

FIG. 2B is an image taken by a monochromatic digital camera of the fluorescent signal from Cy5 dye conjugated to strepavadin associated with a biotin bearing nucleic acid probe to the centromere of human chromosome 11 hybridized to the same sample as FIG. 2A.

FIG. 2C is an image taken by a monochromatic digital camera of the fluorescent signal from Rhodamine conjugated to anti-digoxingenin antibodies associated with a nucleotides incorporated into breaks in the nuclear DNA of the sample as FIG. 2A.

FIG. 2D is a mask created to blank out the regions which display a fluorescent image in both FIG. 2A and FIG. 2C.

FIG. 2E is FIG. 2B with the mask of FIG. 2D overlaid.

FIG. 3A is an image taken by a monochromatic digital camera of the fluorescent signal from DAPI reacted with the nuclear DNA of a sample of breast cancer tissue.

FIG. 3B is an image taken by a monochromatic digital camera of the fluorescent signal from Rhodamine conjugated to anti-digoxingenin antibodies associated with a nucleotides incorporated into breaks in nuclear DNA of the same sample as FIG. 3A.

FIG. 3C is an image taken by a monochromatic digital camera of the fluorescent signal from a green dye conjugated directly to a nucleic acid probe to the centromere of human chromosome 7 hybridized to the same sample as FIG. 3A.

FIG. 3D is an image taken by a monochromatic digital camera of the fluorescent signal from a aqua dye conjugated directly to a nucleic acid probe to the centromere of human chromosome 8 hybridized to the same sample as FIG. 3A.

FIG. 3E is an image taken by a monochromatic digital camera of the fluorescent signal from Cy5 dye conjugated directly to a nucleic acid probe to the centromere of human chromosome 10 hybridized to the same sample as FIG. 3A.

### DETAILED DESCRIPTION

Unless defined otherwise, technical and scientific terms used herein have the same meaning as is commonly understood by one of skill in the art to which this invention belongs. The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced item, and the terms “front”, “back”, “bottom”, and/or “top”, unless otherwise noted, are merely used for convenience of description, and are not limited to any one position or spatial orientation. If ranges are disclosed, the endpoints of all ranges directed to the same component or property are inclusive and independently combinable (e.g., ranges of “up to about 25 wt. %, or, more specifically, about 5 wt. % to about 20 wt. %,” is inclusive of the endpoints and all intermediate values of the ranges of “about 5 wt. % to about 25 wt. %,” etc.). The modifier “about” used in connection with a quantity is inclusive of the stated value and has the meaning dictated by the context (e.g., includes the degree of error associated with measurement of the particular quantity).

The process for improving the accuracy of a fluorescence in-situ hybridization (FISH) assay of a sample of cells, typically a tissue sample, in which there are likely to be cells in apoptosis involves removing such cells from the analysis of the FISH image being analyzed. Such apoptotic cells are first labeled, typically by immunological or nucleic acid tagging, and then the image analysis is conducted without consideration of these tagged cells.

There are a number of techniques known for the labeling of cells in the various stages of apoptosis. Some of these involve detecting the nuclear DNA fragmentation which is a characteristic of apoptosis and others involve detecting cell surface markers characteristic of apoptosis, typically with antibodies, antibody fragments, antibody mimics or receptor proteins specific for such cell surface markers.

Two techniques for the detection of the nuclear DNA fragmentation useful in the present invention are terminal deoxynucleotidyl transferase-mediated dUTP nick end-labeling (TUNEL) and in situ end-labeling (INSEL). These techniques are particularly valuable in more reliably detecting cells in apoptosis than mere cell morphology both because they detect apoptosis earlier than it is apparent in the morphology and because the morphology indicative of apoptosis may be obscured by other morphological effects. Both techniques are well known from the technical literature in the field.

TUNEL labeling is effected by incorporation of labeled nucleotides into the 3′ hydroxyl recessed termini of the DNA breaks characteristic of apoptosis using the enzyme terminal transferase. The incorporated nucleotide may be labeled by a wide variety of techniques. A typical approach is to incorporate a ligand such as fluorescein, biotin or digoxigenin into the nucleotide. If the ligand itself is not capable of yielding a signal, typically fluorescence, it can be reacted with a second moiety such as an appropriate antibody or other receptor which does carry a signal generator after incorporation of the nucleotide into the DNA terminal. Typical of such an approach is the use of a digoxigenin carrying nucleotide with the later reaction with an anti-digoxigenin antibody carrying Rhodamine, or a bromolated nucleotide with the later reaction with an appropriate antibody carrying fluorescein.

INSEL labeling is effected in a similar manner to TUNEL labeling except that the labeled nucleotide is incorporated using the enzyme DNA polymerase I or its Klenow fragment. It is general considered somewhat less sensitive and specific than TUNEL labeling.

Both TUNEL and INSEL labeling require that certain steps be taken in order to have the labeled nucleotides access the nuclear DNA of the cells being analyzed. These steps are well known and included in the instructions accompanying the commercial kits. In general they involve rendering the cell walls of the cells being analyzed permeable to the labeled nucleotide and incorporating enzyme and removing any protein masking by appropriate protein digestion such as with pepsin.

There is an advantage is conducting the TUNEL or INSEL labeling before conducting the FISH assay. The typical procedure for the FISH assay involves denaturing the nuclear DNA of the cells under analysis. This procedure can create DNA breaks which the TUNEL or INSEL procedure would then label giving an inaccurate indication of apoptosis.

One approach is to delay developing the signal from the TUNEL or INSEL labeling until after the FISH assay has been completed. In this case the labeled nucleotides carry a hapten or other binding partner such as biotin. After the cell sample has been treated with the FISH probes the signal from the apoptotic assay is developed by treating the labeled nucleotides with an appropriate developing agent such as an antibody to the hapten or strepavidin which carries a fluorescent moiety. For instance the apoptotic cells could be labeled with a nucleotide which carries a digoxygenin moiety, then the cell sample is exposed to the FISH probes in an appropriate manner and then the cell sample is exposed to the anti-digoxygenin antibody carrying a Rhodamine moiety.

Regardless of whether the apoptotic cells are labeled via DNA breaks or cell surface markers it is important that the signal from the label be distinguishable from that obtained from the FISH assay. This may be achieved in a number of ways. The signal from the apoptosis label may be fluorescence at a readily distinguishable wavelength than that from the FISH assay, the signal generating moiety may be of a type that its signal generating characteristics can be destroyed by appropriate treatment before the FISH assay or an image may be preserved of the signal from apoptotic cells though these latter two approaches requires precise registry of this image with the later image obtained from the FISH assay. Thus there may be an advantage in simultaneously obtaining the signal from both the apoptotic assay and the FISH assay.

The FISH assay is very well known in the field and there are a number of commercial kits available for its performance. In general it involves the use of fluorescently labeled DNA probes to detect certain DNA sequences in the DNA of a cell sample. The cells are appropriately treated to provide the probes access to their DNA and then the probes are hybridized to the target DNA sequences, if they are present. The length of the probes and the conditions of hybridization such as temperature and salt content of the hybridization medium, which conditions are typically referred to as the stringency, are typically adjusted to yield a desired level of specificity.

In addition to the apoptotic assay and the FISH assay it is advantageous to label the total DNA of the cells being evaluated. This facilitates a determination of whether a given apoptotic signal and a given FISH signal are originating from the same cell. Dyes for such labeling such as the intercalating dyes like 4′,6-diamidino-2-phenylindole (DAPI) are well known in the field.

The cell specimens to be analyzed are typically fixed tissue samples. Such tissue specimens are typically fixed in formalin and embedded in paraffin for sectioning with a microtome. The sectioned specimens can then be mounted, typically on microscope slides, and the treated appropriately for the apoptotic and FISH assays. Commonly this involves removal of the paraffin and hydration for the apoptotic assay if it is a TUNEL or INSEL assay. Any such hydration may be reversed before exposure to the FISH probes in order to minimize the quantities of probes needed.

The cell or tissue samples may be of any type in which it is likely that some of the cells will be in apoptosis and the process of the present invention is particularly advantageous when a significant number of cells in the specimen are in apoptosis. One class of such cells is cancer cells taken from a tumor or a leukemia. Another source of such cells might be those which have been subjected to a pharmaceutical undergoing evaluation which is known to cause apoptosis.

The signals obtained from a combined apoptotic assay and FISH assay can be processed in a number of ways. The signals from both assays may be combined into a single image and the image inspected by a trained microscopist who would use the signal from the apoptotic cells to eliminate them from the analysis. Image processing techniques may also be used to exclude apoptotic cells from an evaluation of the FISH assay. In one approach a mask may be created which blanks out any apoptotic cells from the image to be used in evaluating the results from the FISH assay. One way to create such a mask is to construct the apoptotic assay and the FISH assay so that each generates its own distinct fluorescence, typically by fluorescing at wave length substantially different from that displayed by the other assay, and using well known image processing software to blank out the regions associated with apoptotic cells. In this regard the use of an intercalating dye which labels all the nuclear DNA present in the sample can be helpful in defining the region to be associated with any given apoptotic cell.

The evaluation of the FISH assay may be automated. In such a case the software used to conduct the evaluation may be used to eliminate from the analysis any regions associated with apoptotic cells. For instance the software may create and evaluate a virtual image from which these regions have been eliminated so that the presence or absence of a FISH signal from a region associated with any apoptotic cell plays no part in the evaluation. Thus the evaluation will not be subject to errors because an apoptotic cell failed to generate a FISH signal or generated a superious FISH signal because of the fragmentation of its nuclear DNA.

The failure to observe an expected FISH signal from an apoptotic cell can be quite significant to the accurate interpretation of the assay. For instance, in the evaluation of a FISH assay of cancer cells to detect the deletion of a tumor suppressor gene, say the p53 gene, it is important to disregard the absence of signal from cells in apoptosis as the lack of signal is an unreliable indicator. It may just be due to DNA fragmentation from the apoptosis.

In another instance the failure to observe a FISH signal may be lead to the incorrect conclusion that a condition is absent. For instance, in an assay for the Her2 breast cancer marker the FISH assay may fail to show some gene amplification because some of the cells being interrogated are in apoptosis. In another instance there may be a complete absence of signal because all the cells being interrogated are in apoptosis. For example, in an analysis of a lymphoid cancer the sample procurement process may have inadvertently induced apoptosis.

The process described hereinabove for improving the accuracy of FISH assays may be conveniently implamented using a kit comprising reagents to label the apoptotic cells in said sample; reagents with which to conduct said FISH assay; and instructions directing that the cells in the sample which are apoptotic be eliminated from the evaluation of the sample so that the presence or absence of a FISH signal from these cells plays no part in the evaluation. It may also be conveniently implamented using a signal processing system adapted to generate a corrected signal from a fluorescence in-situ hybridization (FISH) assay of a sample of cells containing apoptotic cells comprising means for reading a signal from labeled apoptotic cells in said sample; means for reading the signal from FISH assay of the sample; and means for eliminating said apoptotic cells from the evaluation of the sample so that the presence or absence of a FISH signal from these cells plays no part in the evaluation. The means for reading both signals may be a digital imaging means such as a digital camara and the means for removing said apoptotic cells from the evaluation of the sample may be software programming which generates a corrected image from which said apoptotic cells have been removed.

### EXAMPLE 1

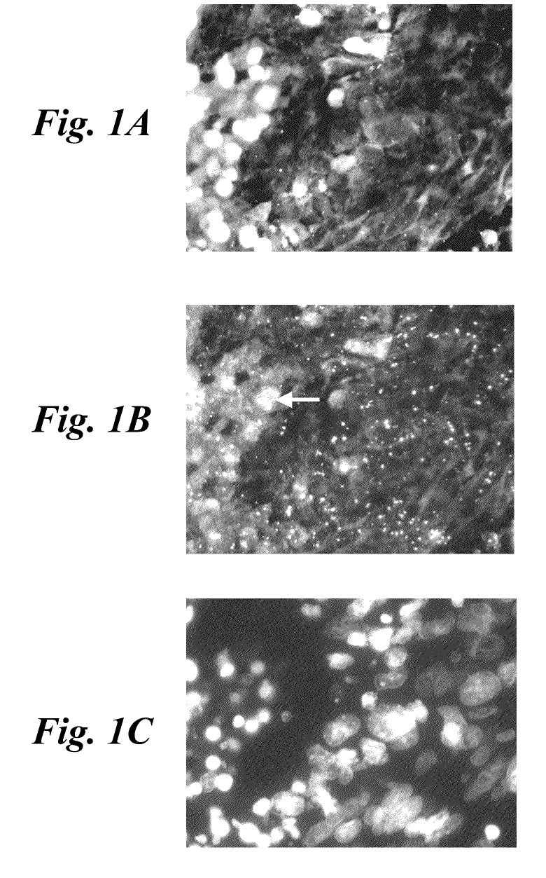

A FISH evaluation of human cancer tissue was made using the techniques of the present application for eliminating error from cells in apoptosis. A xenograph of human colon cancer implemented in nude mice was selected because it was rapidly growing and began running out of nutrients and displaying apoptosis. The following protocol was followed:
1. Slides of xenograft tumor samples were fixed in 10% neutral buffered formalin and processed for paraffin embedding and tissue sectioning. The slides were backed at 65 C for 1 hour. Parafin was further removed from sample sections with Amresco's HistoChoice Clearing Agent (an environmentally friendly xylene alternative) from for 15 minutes.
2. The slides were then processed through a series of alcohol incubations of decreasing concentration of ethanol in water (100, 95. 70, 50%), twice at each concentration for 10 minutes, to hydrate the samples they carried.
3. The samples on the slides are then brought to saline conditions by incubation in PBS solution for 10 minutes.
4. The crosslinked structures produced by formalin fixation were relieved by antigen retrieval method where sample is placed in Sodium Citrate pH 6 in an pressure cooker for 25 minutes (longer times are also suitable) at hi heat and allowed to cool to room temperature (other Heat Pre-treatment methods are also suitable).
5. The samples were then “permeablized” with a brief treatment with PBS containing 0.3% Triton X-100 surfactant.
6. These samples were then digested with Proteinase K enzyme (diluted in PBS for final concentration of 20 ug/ml) for 15 min at Room temperature to remove nuclear protein interference.
7. These samples were then incubated in PBS for 15 minutes and equilibrated in TdT labeling buffer from Chemicon's ApopTag® Red In Situ Apoptosis Detection Kit (Catalogue Number: S7165) according to the kits instructions.
8. These samples were then briefly rinsed with 100 ul reaction buffer and labeled for 1 hr. with terminal deoxytransferase to encorporate labeled nucleotide conjugated to digoxigenin at DNA breaks. In accordance with the kits instructions 77 ul of Reaction buffer mixed with 33 ul of Terminal Deoxytransferase enzyme was applied to these samples, which were then covered with a plastic coverslip and incubated at room temperature. These samples were then incubated in the stop solution from the Chemicon Kit for 10 minutes.
9. Samples were then dehydrated using a 50, 70, 95% ethanol in water series of 10 minute incubations, two at each concentration, and air-dried to reduce the volume of the samples being treated and thus minimize the amount of FISH probes needed.
10. These sample were each then treated with 15 ul of biotin conjugated FISH probes from a Zymed kit (1× working strength from supplier) for the centromere of human chromosome 10 in accordance with the instructions of the kit and a coverslip was sealed over the probe. These samples were heated to 95° C. for 10 minutes and then incubated for 10 hours at 37° C. to allow probes to hybridize to the sample. These samples were then incubated in 2×SSC for 10 minutes, 0.5×SSC for 10 minutes, both at room temperature, then 0.5×SSC at 65° C. for 10 minutes, washed for 5 minutes in 2×SSC, then equilibrated with PBS for 10 minutes.
11. A signal generator was associated with the labeled nucleotides from the TUNEL reaction which detects apoptosis by exposure of these samples to a Rhodamine conjugated anti-digoxigenin antibody provided in the TUNEL kit while simultaneously a different signal generator was associated with the FISH probe for the centromere of human chromosome 10 by exposure to a Cy5 dye-strepavidin conjugate from Jackson Labs. The exposure was to 68 ul incubation buffer and 62 ul antibody solution from the Chemicon TUNEL kit and 1 ul of a Cy5-StrepAvidin conjugate solution from Jackson Labs.
12. These samples were washed, stained with 10 ug/ml DAPI (an intercalating dye which stains nuclear DNA) in PBS to mark the nuclei of the cells in these samples, washed in PBS, and coverslipped with anti-fade mounting media.
13. These samples were then examined in a microscope designed for fluorescence microscopy and coupled to a monochromatic digital camara. The microscope was equipped with the appropriate filters to limit the excitation and emission wavelengths to those appropriate for each the Rhodamine, Cy5 and DAPI. Thus a digital image was created for each of these three markers. FIGS. 1A, 1B and 1C are the fluorescent images from the Rhodamine, Cy5 and DAPI, respectively, at a magnification of [Michael, please insert a value here].
14. Image analysis software was used to create a mask such that only the portion of the Cy5 image not associated with a cell displaying Rhodamine fluorescence was subject to evaluation or scoring. In other words any cell displaying apoptosis was eliminated from evaluation using a combination of the TUNEL signal and the DAPI signal.

### EXAMPLE 2

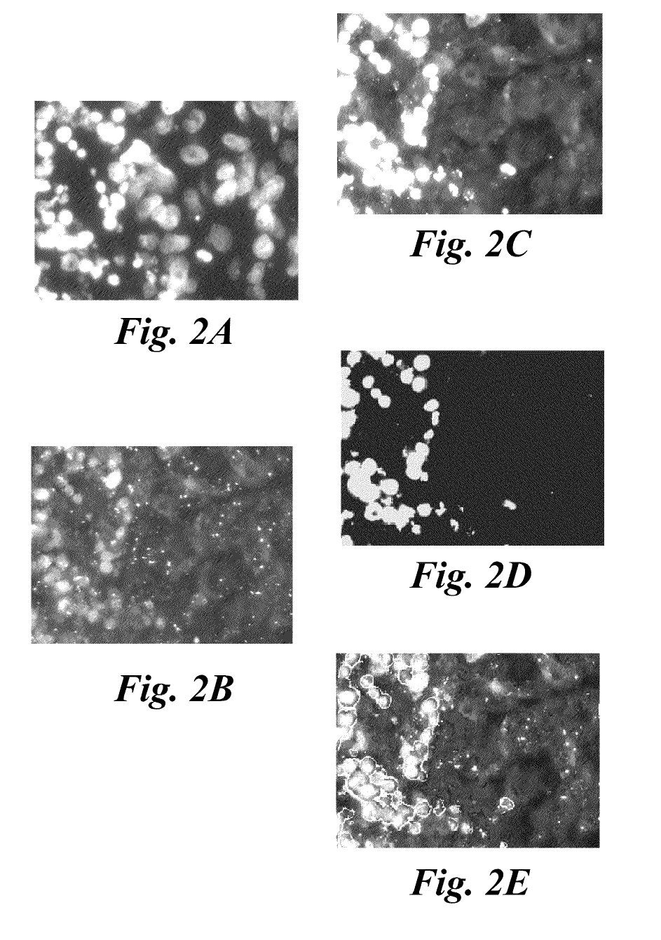

Example 1 was essentially repeated except that the biotin conjugated FISH probe was directed against the centromere of human chromosome 11. FIGS. 2A, 2B and 2C show the digital images from the DAPI, CY5 and Rhodamine. FIG. 2D shows the construction of a mask consisting of an image of the cell nuclei associated with apoptosis as determined from the TUNEL and DAPI analysis, i.e. the mask includes those cell nuclei identified by DAPI which contained a Rhodamine signal. FIG. 2E shows the digital image of the Cy5 FISH signal overlaid with the mask. It is appropriate for evaluation by automated means such as computer scoring or by a skilled microscopist.

### EXAMPLE 3

Example 3 followed a protocol very similar to that of Example 1 except that the probes were directly conjugated to signal generators so that the step of associating the probes with a dye-strepavadin conjugate was unnecessary and was therefore omitted. Furthermore, the sample material human breast cancer tissue instead of a xenograft tumor from nude nice and three different FISH probes were used.

The probes were Poseiden probes from Immunicon that were made synthetically to avoid any DNA repeats in their sequences. These probes represent a departure from earlier technology such as used in the Zymed probes of Examples 1 and 2 in which DNA repeats are cared for by including unlabeled probes to the repeat sequence in the probe reagent.

The probes were directed against the centromeres of human chromosomes 7, 8 and 10. The probes were directly conjugated with a green dye, an aqua dye and Cy5, respectively.

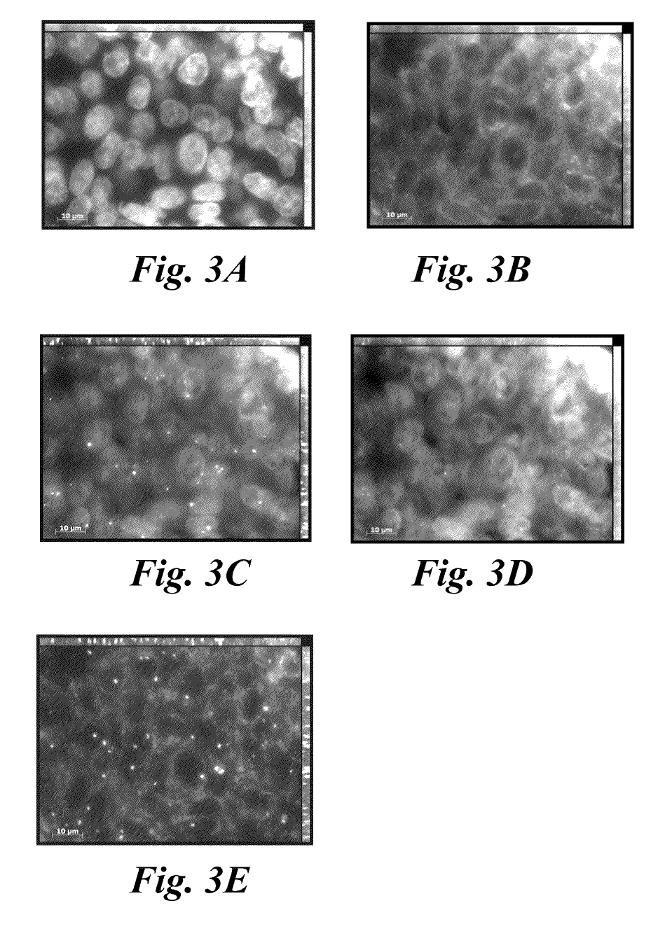

Appropriate filters for each of these three dyes as well as filters appropriate for both the DAPI and the Rhodamine were employed in the imaging. FIG. 3A is the digital image from DAPI while FIG. 3B is that of Rhodamine. FIGS. 3C, 3D and 3E are the digital images from the green, aqua and Cy5 dyes, respectively.

From FIG. 3B it can be seen that the region under examination is essentially free from cells in apoptosis. Thus a mask to overlay FIGS. 3C, 3D and 3E is unnecessary as there are essentially no cells in apoptosis to be eliminated from the evaluation. Thus the presence or absence of a FISH signal from the entire region under examination can be considered in the evaluation.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A process for improving the accuracy of a fluorescence in-situ hybridization (FISH) assay of a sample of cells in which there is likely to be a substantial number of cells in apoptosis comprising:
a) labeling the apoptotic cells involved in the assay;
b) conducting the FISH assay;
c) by manual or automated means removing from the analysis the cells that are in apoptosis as indicated by a signal from the apoptosis assay.

2. The process of claim 1 wherein the apoptotic cells are labeled by attachment of a labeling moiety to breaks in their nuclear DNA.

3. The process of claim 2 wherein the apoptotic cells are labeled before the FISH assay is conducted.

4. The process of claim 3 wherein the apoptotic cells are labeled by in situ end-labeling using polymerase (ISEL) or by terminal deoxynucleotidyl transferase labeling (TUNEL).

5. The process of claim 4 wherein the apoptotic cells are labeled by TUNEL using nucleotides carrying haptens which are subsequently recognized by antibodies conjugated to a signal generator.

6. The process of claim 5 wherein the FISH assay is conducted before the introduction of said conjugated antibodies.

7. The process of claim 1 wherein the cell sample is drawn from a cancer.

8. The process of claim 7 wherein the cell sample is drawn from a tumor.

9. The process of claim 7 wherein the cell sample is drawn from a leukemia.

10. The process of claim 1 wherein the signals from both the apoptotic assay and the FISH assay are captured by an automated signal processing system.

11. The process of claim 10 wherein said signals are processed such that apoptotic cells are eliminated from an evaluation of the results of the FISH assay.

12. A process for conducting a fluorescence in-situ hybridization (FISH) assay of a sample of cells comprising:
d) labeling the apoptotic cells involved in the assay;
e) conducting the FISH assay;
f) by manual or automated means eliminating from evaluation the regions of sample associated cells which are in apoptosis as indicated by a signal from the apoptosis assay; and
g) evaluating the FISH signal from the remaining regions.

13. The process of claim 12 wherein the regions for elimination are identified as the regions simultaneously yielding a signal from the apoptosis assay and an assay for nuclear DNA in general.

14. The process of claim 13 wherein the nuclear DNA of the cells undergoing examination is labeled with a signal generator to define the nuclei of said cells and any so defined nucleus that also exhibits a signal indicative of apoptosis is eliminated from evaluation.

15. The process of claim 14 wherein the apoptotic cells are labeled by the incorporation of labeled nucleotides into breaks in their nuclear DNA.

16. The process of claim 15 wherein the labeled nucleotide is incorporated by in situ end-labeling using polymerase (ISEL) or by terminal deoxynucleotidyl transferase labeling (TUNEL).

17. The process of claim 16 wherein the labeled nucleotides are incorporated before the hybridization step of the FISH assay.

18. A kit for the dual apoptosis-fluorescence in-situ hybridization (FISH) assay of a sample of cells comprising:
h) reagents to label the apoptotic cells in said sample;
i) reagents with which to conduct said FISH assay; and
j) instructions directing that the cells in the sample which are apoptotic be eliminated from the evaluation of the sample so that the presence or absence of a FISH signal from these cells plays no part in the evaluation.

19. A signal processing system adapted to generate a corrected signal from a fluorescence in-situ hybridization (FISH) assay of a sample of cells containing apoptotic cells comprising;
k) means for reading a signal from labeled apoptotic cells in said sample;
l) means for reading the signal from FISH assay of the sample; and
m) means for eliminating said apoptotic cells from the evaluation of the sample so that the presence or absence of a FISH signal from these cells plays no part in the evaluation.

20. The system of claim 19 wherein:
n) the signal from the apoptotic cells is a light signal;
o) the means for reading both said signals is a digital imaging means; and
p) the means for removing said apoptotic cells from the evaluation of the sample comprises programming which generates a corrected image from which said apoptotic cells have been removed.
